(19) United States
(12) Patent Application Publication
(10) Pub. No.: US 2009/0143640 A1
(43) Pub. Date: Jun. 4, 2009

(54) COMBINATION IMAGING AND TREATMENT ASSEMBLIES

(75) Inventors: Vahid SAADAT, Atherton, CA (US); Ruey-Feng PEH, Mountain View, CA (US); Zachary J. MALCHANO, San Francisco, CA (US); David MILLER, Cupertino, CA (US); Chris A. ROTHE, San Mateo, CA (US); Juan Diego PEREA, Gilroy, CA (US)

Correspondence Address: LEVINE BAGADE HAN LLP, 2483 EAST BAYSHORE ROAD, SUITE 100, PALO ALTO, CA, 94303, US

(73) Assignee: Voyage Medical, Inc., Campbell, CA (US)

(21) Appl. No.: 12/323,281

(22) Filed: Nov. 25, 2008

Related U.S. Application Data
(60) Provisional application No. 60/990,231, filed on Nov. 26, 2007.

(51) Int. Cl. A61B 1/06; A61B 1/00; A61B 18/14

(52) U.S. Cl. 600/104; 600/160; 606/41

(57) ABSTRACT

Combination imaging and treatment assemblies are described herein which may utilize a deployment catheter in combination with an endoscopic system. The combined system comprises an open architecture to modularly incorporate any number of imaging devices (such as optical fiber, CMOS or CCD endoscopes) to provide high resolution optical images of tissue within an opaque environment. Additional variations may include an imaging hood or balloon member incorporated upon an endoscope or advanced through an endoscope working channel to visualize and treat tissue through blood.

### CROSS-REFERENCE TO RELATED APPLICATIONS

This application claims the benefit of priority to U.S. Provisional Application No. 60/990,231, filed Nov. 26, 2007, which is incorporated herein by reference in its entirety.

### FIELD OF THE INVENTION

The present invention relates Generally to medical devices used for accessing, visualizing, and/or treating regions of tissue within a body. More particularly, the present invention relates to methods and apparatus of a tissue visualization and treatment device that is able to provide high resolution digital optical images of tissue within a body.

### BACKGROUND OF THE INVENTION

Conventional devices for visualizing interior regions of a body lumen are known. For example, ultrasound devices have been used to produce images from within a body in vivo. Ultrasound has been used both with and without contrast agents, which typically enhance ultrasound-derived images.

Other conventional methods have utilized catheters or probes having position sensors deployed within the body lumen, such as the interior of a cardiac chamber. These types of positional sensors are typically used to determine the movement of a cardiac tissue surface or the electrical activity within the cardiac tissue. When a sufficient number of points have been sampled by the sensors, a “map” of the cardiac tissue may be generated.

Another conventional device utilizes an inflatable balloon which is typically introduced intravascularly in a deflated state and then inflated against the tissue region to be examined. Imaging is typically accomplished by an optical fiber or other apparatus such as electronic chips for viewing the tissue through the membrane(s) of the inflated balloon. Moreover, the balloon must generally be inflated for imaging. Other conventional balloons utilize a cavity or depression formed at a distal end of the inflated balloon. This cavity or depression is pressed against the tissue to be examined and is flushed with a clear fluid to provide a clear pathway through the blood.

However, such imaging balloons have many inherent disadvantages. For instance, such balloons generally require that the balloon be inflated to a relatively large size which may undesirably displace surrounding tissue and interfere with fine positioning of the imaging system against the tissue. Moreover, the working area created by such inflatable balloons are generally cramped and limited in size. Furthermore, inflated balloons may be susceptible to pressure changes in the surrounding fluid. For example, if the environment surroundings the inflated balloon undergoes pressure changes, e.g., during systolic and diastolic pressure cycles in a beating heart, the constant pressure change may affect the inflated balloon volume and its positioning to produce unsteady or undesirable conditions for optimal tissue imaging. Additionally, imaging balloons are subject to producing poor or blurred tissue images if the balloon is not firmly pressed against the tissue surface because of intervening blood between the balloon and tissue.

Accordingly, these types of imaging modalities are generally unable to provide desirable images useful for sufficient diagnosis and therapy of the endoluminal structure, due in part to factors such as dynamic forces generated by the natural movement of the heart. Moreover, anatomic structures within the body can occlude or obstruct the image acquisition process. Also, the presence and movement of opaque bodily fluids such as blood generally make in vivo imaging of tissue regions within the heart difficult.

Other external imaging modalities are also conventionally utilized. For example, computed tomography (CT) and magnetic resonance imaging (MRI) are typical modalities which are widely used to obtain images of body lumens such as the interior chambers of the heart. However, such imaging modalities fail to provide real-time imaging for intra-operative therapeutic procedures. Fluoroscopic imaging, for instance, is widely used to identify anatomic landmarks within the heart and other regions of the body. However, fluoroscopy fails to provide an accurate image of the tissue quality or surface and also fails to provide for instrumentation for performing tissue manipulation or other therapeutic procedures upon the visualized tissue regions. In addition, fluoroscopy provides a shadow of the intervening tissue onto a plate or sensor when it may be desirable to view the intraluminal surface of the tissue to diagnose pathologies or to perform some form of therapy on it.

Thus, a tissue imaging system which is able to provide real-time in vivo images of tissue regions within body lumens such as the heart through opaque media such as blood and which also provide instruments for therapeutic procedures upon the visualized tissue are desirable.

### SUMMARY OF THE INVENTION

A tissue imaging and manipulation apparatus that may be utilized for procedures within a body lumen, such as the heart, in which visualization of the surrounding tissue is made difficult, if not impossible, by medium contained within the lumen such as blood, is described below. Generally, such a tissue imaging and manipulation apparatus comprises an optional delivery catheter or sheath through which a deployment catheter and imaging hood may be advanced for placement against or adjacent to the tissue to be imaged.

The deployment catheter may define a fluid delivery lumen therethrough as well as an imaging lumen within which an optical imaging fiber or assembly may be disposed for imaging tissue. When deployed, the imaging hood may be expanded into any number of shapes, e.g., cylindrical, conical as shown, semi-spherical, etc., provided that an open area or field is defined by the imaging hood. The open area is the area within which the tissue region of interest may be imaged. The imaging hood may also define an atraumatic contact lip or edge for placement or abutment against the tissue region of interest. Moreover, the distal end of the deployment catheter or separate manipulatable catheters may be articulated through various controlling mechanisms such as push-pull wires manually or via computer control

The deployment catheter may also be stabilized relative to the tissue surface through various methods. For instance, inflatable stabilizing balloons positioned along a length of the catheter may be utilized, or tissue engagement anchors may be passed through or alone the deployment catheter for temporary engagement of the underlying tissue.

In operation, after the imaging hood has been deployed, fluid may be pumped at a positive pressure through the fluid delivery lumen until the fluid fills the open area completely and displaces any blood from within the open area. The fluid may comprise any biocompatible fluid., e.g., saline, water, plasma, Fluorinert™, etc., which is sufficiently transparent to allow for relatively undistorted visualization through the fluid. The fluid may be pumped continuously or intermittently to allow for image capture by an optional processor which may be in communication with the assembly.

In an exemplary variation for imaging tissue surfaces within a heart chamber containing blood. the tissue imaging and treatment system may generally comprise a catheter body having a lumen defined therethrough, a visualization element disposed adjacent the catheter body, the visualization element having a field of view, a transparent fluid source in fluid communication with the lumen, and a barrier or membrane extendable from the catheter body to localize, between the visualization element and the field of view, displacement of blood by transparent fluid that flows from the lumen, and an instrument translatable through the displaced blood for performing any number of treatments upon the tissue surface within the field of view. The imaging hood may be formed into any number of configurations and the imaging assembly may also be utilized with any number of therapeutic tools which may be deployed through the deployment catheter.

Another variation of a tissue imaging and treatment assembly may include an endoscope for use in combination with the deployment catheter. Because the assembly may receive an endoscope through a lumen defined therethrough, the endoscope may provide imaging functionality as well as optional steering or articulation capabilities to the assembly when in use in a patient. This allows for a system to be assembled which may be optionally disposed after a single use or limited number of uses. Accordingly, the assembly may generally comprise the deployment catheter which defines a lumen therethrough extending from a hub. The hood the may be positioned upon the distal end of the deployment catheter and may optionally include an electrode assembly, e.g., mapping, pacing, and/or ablation electrodes, positioned upon the hood. The hood may be actuated between its low-profile delivery configuration and extended and deployed configuration via an actuating, mechanism such as a hood retraction control which may be located along the catheter. Aside from the use of hood structures, other imaging and treatment structures such as a double-layered balloon may be utilized with any of the deployment catheter devices described herein. An optional fluid irrigation port may also extend from the hub to fluidly couple a reservoir, which may hold the clearing fluid (or other fluids), to the hood for providing the purging fluid. Moreover, the assembly may also include an interface seal along the hub to provide a seal when an endoscope shaft is advanced through the hub and distally through the catheter.

As previously mentioned, an endoscope may be inserted into the catheter system to optionally provide imaging functionality. In addition to the endoscope, the deployment catheter assembly may be further utilized with an introducer sheath through which the catheter and endoscope may be advanced. The introducer sheath may further include a fluid irrigation port extending from the sheath for coupling to a fluid reservoir or for providing access to other instruments into the patient body. An additional endoscope handle interface may be attached to the hub for facilitating coupling and de-coupling to the endoscope handle. The interface may be configured to receive any number of endoscope handles for securely retaining and maintaining its position relative to the catheter when in use.

In addition to the steering capabilities of the deployment catheter, the hood may utilize additional features such as a guidewire which may pass through a rapid exchange port defined along the hood. Yet another feature which may be optionally incorporated with the hood may include a ferromagnetic ring for magnetic steering of the hood utilizing systems such as the Niobe® magnetic navigation system by Stereotaxis, Inc.

Another feature which may be optionally incorporated with the deployment catheter includes an advancement control, which may be positioned proximal to the catheter. The advancement control may function as an optical zoom feature such that when the control is rotated about its longitudinal axis, the length of the catheter shaft may be varied relative to the length of the endoscope shaft which in turn changes the relative position of the endoscope lens with respect to the imaging hood and varies the distance between the lens and the imaged tissue.

Turning now to other examples and features which may be utilized with tile devices and methods described herein, the hood may be coupled directly to an endoscope distal end rather than utilizing a separate deployment catheter. A hood connector member may be attached to a distal portion of an endoscope shaft via a securement portion which defines a locking feature for coupling at least temporarily to the hood, e.g., threaded as shown, tabs, screw-on coupler, male-female snap fits, elastic bands, clamps, friction lock, Velcro® patches, adhesive, etc. In this manner, the securement portion may be fitted upon any endoscope distal end by engaging with the hood connector located proximal to the hood in a complementary engagement. Any cables or connectors, such as wires attached to any electrodes or imaging sensors located within or along the hood, leading from the hood may be passed through the endoscope working lumen for coupling to their appropriate connections outside the patient body.

In yet another variation, a hood may be positioned upon a fluid support member and advanced through an endoscope working lumen while maintaining a low-profile delivery configuration. Upon advancement past the lumen opening, the hood may automatically expand or be actuated to expand into its deployed profile such that a proximal hood opening is defined through the hood. Once expanded, the support member may be proximally withdrawn to pull the hood into firm contact against the distal end of endoscope shaft such that the opening at least partially encircles the imaging element of the endoscope. The interior of the hood may accordingly be purged of any blood by introducing the clearing fluid either through the member and/or endoscope lumen for visualizing the underlying tissue.

### BRIEF DESCRIPTION OF THE DRAWINGS

FIG. 1A shows a side view of one variation of a tissue imaging apparatus during deployment from a sheath or delivery catheter.

FIG. 1B shows the deployed tissue imaging apparatus of FIG. 1A having an optionally expandable hood or sheath attached to an imaging and/or diagnostic catheter.

FIG. 1C shows an end view of a deployed imaging apparatus.

FIGS. 2A and 2B show one example of a deployed tissue imager positioned against or adjacent to the tissue to be imaged and a flow of fluid, such as saline, displacing blood from within the expandable hood.

FIGS. 3A and 3B show examples of various visualization imagers which may be utilized within or along the imaging hood.

FIGS. 4A and 4B show perspective and end views, respectively, of an imaging hood having at least one layer of a transparent elastomeric membrane over the distal opening of the hood.

FIGS. 5A and 5B show perspective and end views, respectively, of an imaging hood which includes a membrane with an aperture defined therethrough and a plurality of additional openings defined over the membrane surrounding the aperture.

FIG. 6 illustrates an assembly view of one example of a visualization system configured with a grounding pad for ablation treatment.

FIG. 7 illustrates an assembly view of another example of a visualization system configured for visualized ablation while viewed upon a monitor.

FIG. 8 illustrates a perspective view of another variation of a visualization and catheter treatment system which may be utilized with an endoscopic assembly.

FIG. 9A illustrates a perspective view of an introducer sheath and endoscopic assembly which may be equipped with a high-resolution digital imaging system.

FIG. 9B illustrates a perspective view of the endoscope inserted into the visualization and treatment catheter.

FIG. 10 illustrates a perspective view of a retracted hood for insertion into an introducer sheath.

FIG. 11 illustrates an assembly view of a visualization and treatment system advanced intravascularly into a patient's heart for diagnosis and/or treatment.

FIG. 12 illustrates an assembly view of another variation of a visualization and catheter treatment system which may be coupled to an endoscope.

FIGS. 13A and 13B show cross-sectional side and perspective views, respectively, of a variation configured for placement over an endoscope.

FIG. 14 illustrates a perspective view of a system having an endoscope positioned therethrough while actively and passively articulated along different planes.

FIG. 15 illustrates a perspective view of a system secured to a handle of an endoscope.

FIG. 16 illustrates perspective and side views of an optional image adjustment mechanism.

FIGS. 17A and 17B illustrate perspective and side views, respectively, of another variation where a hood may be secured to an endoscope distal end.

FIG. 18 shows an assembly view of another variation where an imaging hood may be attached to a distal end of an endoscope.

FIGS. 19A to 19C illustrate cross-sectional side views of another variation of an imaging apparatus configured as a double-layered balloon having one or more apertures or openings.

FIGS. 20A and 20B illustrate side views of a double-layered balloon assembly coupled to an endoscope and to a visualization and catheter treatment system, respectively.

FIGS. 21A to 21C illustrate another variation where a collapsed hood may be advanced through a working lumen of an endoscope and deployed for use in a patient.

FIGS. 22A and 22B illustrate side views of a double-layered balloon assembly and a hood assembly coupled to a fluid lumen for advancement through a working lumen of an endoscope.

### DETAILED DESCRIPTION OF THE INVENTION

A tissue-imaging and manipulation apparatus described herein is able to provide real-time images in vivo of tissue regions within a body lumen such as a heart, which is filled with blood flowing dynamically therethrough and is also able to provide intravascular tools and instruments for performing various procedures upon the imaged tissue regions. Such an apparatus may be utilized for many procedures, e.g., facilitating transseptal access to the left atrium, cannulating the coronary sinus, diagnosis of valve regurgitation/stenosis, valvuloplasty, atrial appendage closure, arrhythmogenic focus ablation, among other procedures.

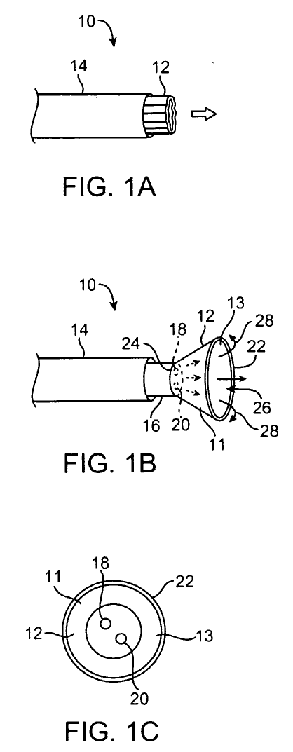

One variation of a tissue access and imaging apparatus is shown in the detail perspective views of FIGS. 1A to 1C. As shown in FIG. 1A, tissue imaging and manipulation assembly 10 may be delivered intravascularly through the patient's body in a low-profile configuration via a delivery catheter or sheath 14. In the case of treating tissue, it is generally desirable to enter or access the left atrium while minimizing trauma to the patient. To non-operatively effect such access, one conventional approach involves puncturing the intra-atrial septum from the right atrial chamber to the left atrial chamber in a procedure commonly called a transseptal procedure or septostomy. For procedures such as percutaneous valve repair and replacement, transseptal access to the left atrial chamber of the heart may allow for larger devices to be introduced into the venous system than can generally be introduced percutaneously into the arterial system.

When the imaging and manipulation assembly 10 is ready to be utilized for imaging tissue, imaging hood 12 may be advanced relative to catheter 14 and deployed from a distal opening of catheter 14, as shown by the arrow. Upon deployment, imaging hood 12 may be unconstrained to expand or open into a deployed imaging configuration, as shown in FIG. 1B. Imaging hood 12 may be fabricated from a variety of pliable or conformable biocompatible material including but not limited to, e.g., polymeric, plastic, or woven materials. One example of a woven material is Kevlar® (E. I. du Pont de Nemours, Wilmington, Del.), which is an aramid and which can be made into thin, e.g., less than 0.001 in., materials which maintain enough integrity for such applications described herein. Moreover, the imaging hood 12 may be fabricated from a translucent or opaque material and in a variety of different colors to optimize or attenuate any reflected lighting from surrounding fluids or structures, i.e., anatomical or mechanical structures or instruments. In either case, imaging hood 12 may be fabricated into a uniform structure or a scaffold-supported structure, in which case a scaffold made of a shape memory alloy, such as Nitinol, or a spring steel, or plastic, etc., may be fabricated and covered with the polymeric, plastic, or woven material. Hence, imaging hood 12 may comprise any of a wide variety of barriers or membrane structures, as may generally be used to localize displacement of blood or the like from a selected volume of a body lumen or heart chamber. In exemplary embodiments, a volume within an inner surface 13 of imaging hood 12 will be significantly less than a volume of the hood 12 between inner surface 13 and outer surface 11.

Imaging hood 12 may be attached at interface 24 to a deployment catheter 16 which may be translated independently of deployment catheter or sheath 14. Attachment of interface 24 may be accomplished through any number of conventional methods. Deployment catheter 16 may define a fluid delivery lumen 18 as well as an imaging lumen 20 within which an optical imaging fiber or assembly may be disposed for imaging tissue. When deployed, imaging hood 12 may expand into any number of shapes, e.g., cylindrical, conical as shown, semi-spherical, etc., provided that an open area or field 26 is defined by imaging hood 12. The open area 26 is the area within which the tissue region of interest may be imaged. Imaging hood 12 may also define an atraumatic contact lip or edge 22 for placement or abutment against the tissue region of interest. Moreover, the diameter of imaging hood 12 at its maximum fully deployed diameter, e.g., at contact lip or edge 22, is typically greater relative to a diameter of the deployment catheter 16 (although a diameter of contact lip or edge 22 may be made to have a smaller or equal diameter of deployment catheter 16). For instance, the contact edge diameter may range anywhere from 1 to 5 times (or even greater, as practicable) a diameter of deployment catheter 16. FIG. 1C shows an end view of the imaging hood 12 in its deployed configuration. Also shown are the contact lip or edge 22 and fluid delivery lumen 18 and imaging lumen 20.

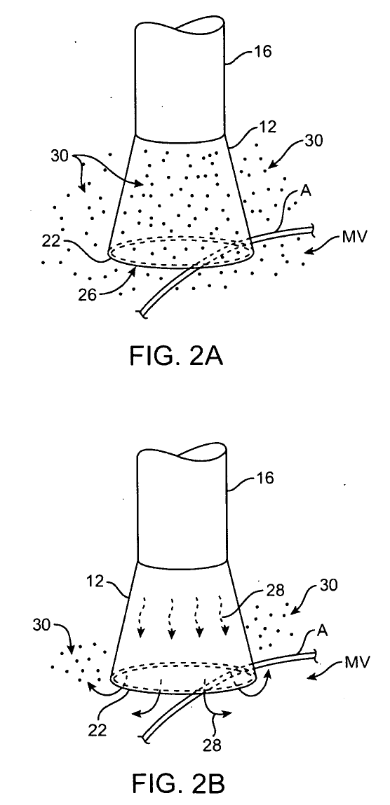

As seen in the example of FIGS. 2A and 2B, deployment catheter 16 may be manipulated to position deployed imaging hood 12 against or near the underlying tissue region of interest to be imaged, in this example a portion of annulus A of mitral valve MV within the left atrial chamber. As the surrounding blood 30 flows around imaging hood 12 and within open area 26 defined within imaging hood 12, as seen in FIG. 2A, the underlying annulus A is obstructed by the opaque blood 30 and is difficult to view through the imaging lumen 20. The translucent fluid 28, such as saline, may then be pumped through fluid delivery lumen 18, intermittently or continuously, until the blood 30 is at least partially, and preferably completely, displaced from within open area 26 by fluid 28, as shown in FIG. 2B.

Although contact edge 22 need not directly contact the underlying tissue, it is at least preferably brought into close proximity to the tissue such that the flow of clear fluid 28 from open area 26 may be maintained to inhibit significant backflow of blood 30 back into open area 26. Contact edge 22 may also be made of a soft elastomeric material such as certain soft grades of silicone or polyurethane, as typically known, to help contact edge 22 conform to an uneven or rough underlying anatomical tissue surface. Once the blood 30 has been displaced from imaging hood 12, an image may then be viewed of the underlying tissue through the clear fluid 30. This image may then be recorded or available for real-time viewing for performing a therapeutic procedure. The positive flow of fluid 28 may be maintained continuously to provide for clear viewing of the underlying tissue. Alternatively, the fluid 28 may be pumped temporarily or sporadically only until a clear view of the tissue is available to be imaged and recorded, at which point the fluid flow 28 may cease and blood 30 may be allowed to seep or flow back into imaging hood 12. This process may be repeated a number of times at the same tissue region or at multiple tissue regions.

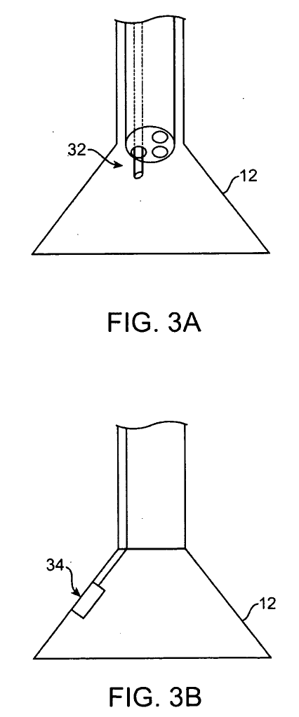

FIG. 3A shows a partial cross-sectional view of an example where one or more optical fiber bundles 32 may be positioned within the catheter and within imaging hood 12 to provide direct in-line imaging of the open area within hood 12. FIG. 3B shows another example where an imaging element 34 (e.g., CCD or CMOS electronic imager) may be placed along an interior surface of imaging hood 12 to provide imaging of the open area such that the imaging element 34 is off-axis relative to a longitudinal axis of the hood 12, as described in further detail below. The off-axis position of element 34 may provide for direct visualization and uninhibited access by instruments from the catheter to the underlying tissue during treatment.

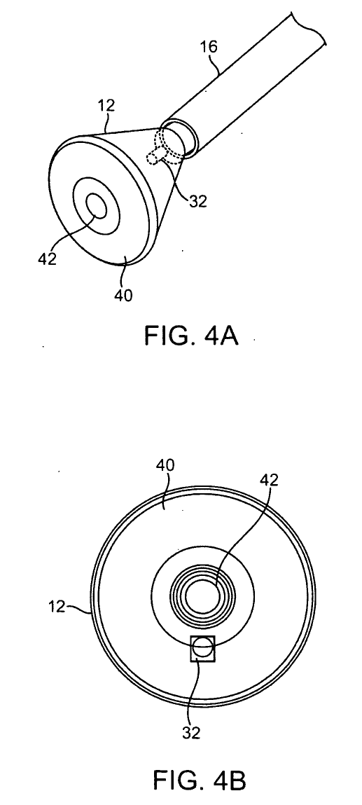

In utilizing the imaging hood 12 in any one of the procedures described herein, the hood 12 may have an open field which is uncovered and clear to provide direct tissue contact between the hood interior and the underlying tissue to effect any number of treatments upon the tissue, as described above. Yet in additional variations, imaging hood 12 may utilize other configurations. An additional variation of the imaging hood 12 is shown in the perspective and end views, respectively, of FIGS. 4A and 4B, where imaging hood 12 includes at least one layer of a transparent elastomeric membrane 40 over the distal opening of hood 12. An aperture 42 having a diameter which is less than a diameter of the outer lip of imaging hood 12 may be defined over the center of membrane 40 where a longitudinal axis of the hood intersects the membrane such that the interior of hood 12 remains open and in fluid communication with the environment external to hood 12. Furthermore, aperture 42 may be sized, e.g., between 1 to 2 mm or more in diameter and membrane 40 can be made from any number of transparent elastomers such as silicone, polyurethane, latex, etc. such that contacted tissue may also be visualized through membrane 40 as well as through aperture 42.

Aperture 42 may function generally as a restricting passageway to reduce the rate of fluid out-flow from tile hood 12 when the interior of the hood 12 is infused with tile clear fluid through which underlying tissue regions may be visualized. Aside from restricting out-flow of clear fluid from within hood 12, aperture 42 may also restrict external surrounding fluids from entering hood 12 too rapidly. The reduction in the rate of fluid out-flow from the hood and blood in-flow into the hood may improve visualization conditions as hood 12 may be more readily filled with transparent fluid rather than being filled by opaque blood which may obstruct direct visualization by the visualization instruments.

Moreover, aperture 42 may be aligned with catheter 16 such that any instruments (e.g., piercing instruments, guidewires, tissue engagers, etc.) that are advanced into the hood interior may directly access the underlying tissue uninhibited or unrestricted for treatment through aperture 42. In other variations wherein aperture 42 may not be aligned with catheter 16, instruments passed through catheter 16 may still access the underlying tissue by simply piercing through membrane 40.

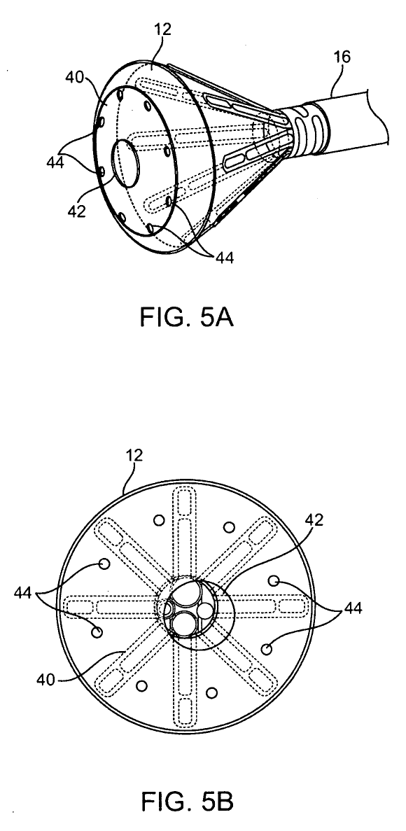

In an additional variation, FIGS. 5A and 5B show perspective and end views, respectively, of imaging hood 12 which includes membrane 40 with aperture 42 defined therethrough, as described above. This variation includes a plurality of additional openings 44 defined over membrane 40 surrounding aperture 42. Additional openings 44 may be uniformly sized, e.g., each less than 1 mm in diameter, to allow for the out-flow of the translucent fluid therethrough when in contact against the tissue surface. Moreover, although openings 44 are illustrated as uniform in size, the openings may be varied in size and their placement may also be non-uniform or random over membrane 40 rather than uniformly positioned about aperture 42 in FIG. 5B. Furthermore, there are eight openings 44 shown in the figures although fewer than eight or more than eight openings 44 may also be utilized over membrane 40.

Additional details of tissue imaging and manipulation systems and methods which may be utilized with apparatus and methods described herein are further described, for example, in U.S. patent application Ser. No. 11/259,498 filed Oct. 25, 2005 (U.S. Pat. Pub. No. 2006/0184048 A1); Ser. No. 11/763,399 filed Jun. 14, 2007 (U.S. Pat. Pub. No. 2007/0293724 A1); and also in Ser. No. 11/828,267 filed Jul. 25, 2007 (U.S. Pat. Pub. No. 2008/0033290 A1), and Ser. No. 11/775,837 filed Jul. 10, 2007 (U.S. Pat. Pub. No. 2008/0009747 A1) each of which is incorporated herein by reference in its entirety.

In treating tissue regions which are directly visualized, as described above, treatments utilizing electrical energy may be employed to ablate the underlying visualized tissue. Many ablative systems typically employ electrodes arranged in a monopolar configuration where a single electrode is positioned proximate to or directly against the tissue to be treated within the patient body and a return electrode is located external to the patient body. In other variations, biopolar configurations may be utilized.

In particular, such assemblies, apparatus, and methods may be utilized for treatment of various conditions, e.g., arrhythmias, through ablation under direct visualization. 30 Details of examples for the treatment of arrhythmias under direct visualization which may be utilized with apparatus and methods described herein are described, for example, in U.S. patent application Ser. No. 11/775,819 filed Jul. 10, 2007 (U.S. Pat. Pub. No. 2008/0015569 Al), which is incorporated herein by reference in its entirety. Variations of the tissue imaging and manipulation apparatus may be configured to facilitate the application of bipolar energy delivery, such as radio-frequency (RF) ablation, to an underlying target tissue for treatment in a controlled manner while directly visualizing the tissue during the bipolar ablation process as well as confirming (visually and otherwise) appropriate treatment thereafter.

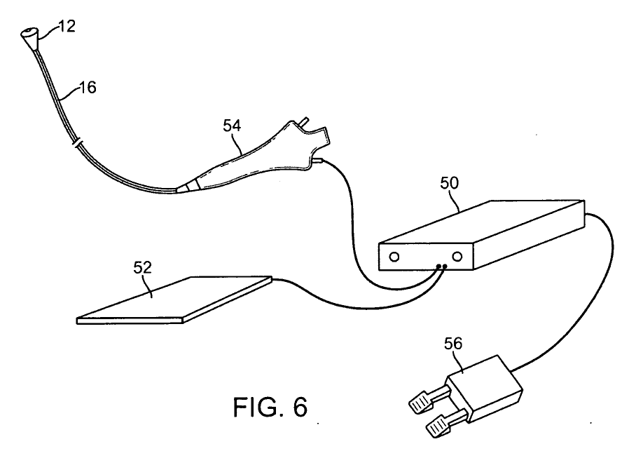

As illustrated in the assembly view of FIG. 6, hood 12 and deployment catheter 16 may be coupled to handle 54, through which the electrode may be coupled to the energy generator 50. The example illustrated shows a monopolar ablation configuration and thus includes grounding plate 52 also electrically coupled to generator 50. A separate actuation assembly 56, e.g., foot pedal, may also be electrically coupled to generator 50 to allow for actuation of the ablation energy. Upon filling the hood 12 with saline and obtaining a clear view of the tissue region of interest, the RF ablation energy generator 50 can be activated via actuation assembly 56 to initiate the flow of electrical currents to be transmitted from the generator 50 and through an ablation probe instrument, or through the purging fluid itself (e.g., saline) via an electrode to electrically charge the saline within the imaging hood 12, or through one or more electrodes positioned along or within the hood 12.

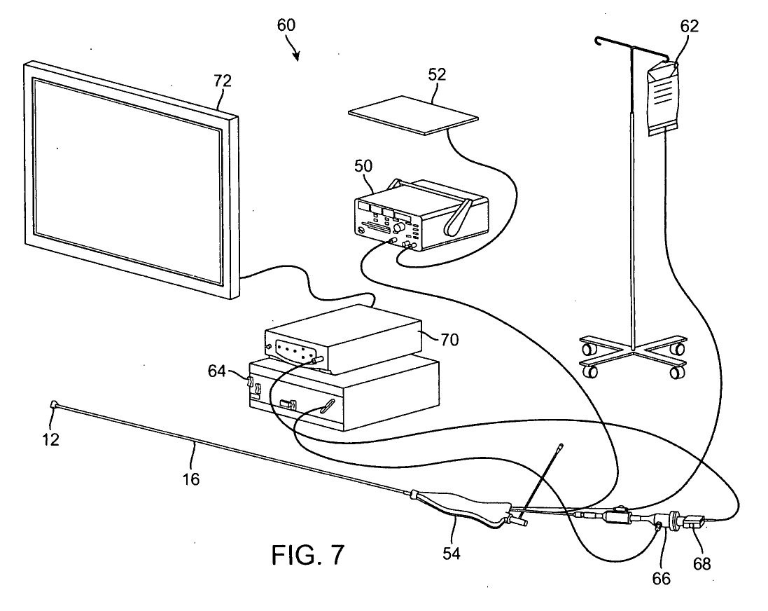

As the assembly allows for ablation of tissue directly visualized through hood 12, FIG. 7 illustrates an example of a system configured for enabling dual visualization and ablation. As shown in ablation assembly 60, hood 12 and deployment catheter 16 are coupled to handle 54, as previously described. Fluid reservoir 62, shown in this example as a saline-filled bag reservoir, may be attached through handle 54 to provide the clearing fluid and/or ablation medium. An optical imaging assembly 66 coupled to an imaging element positioned within or adjacent to hood 12 may extend proximally through handle 54 and be coupled to imagine processor assembly 64 for processing the images detected within hood 12. Assembly may also be coupled to a video receiving assembly 68 for receiving images from the optical imaging assembly 66. The video receiving assembly 68 may in turn be coupled to video processor assembly 70 which may process the detected images within hood 12 for display upon video display 72. Also shown are grounding plate 52 and ablation energy generator 50 which is coupled to ablation electrode within or proximate to hood 12, as previously described.

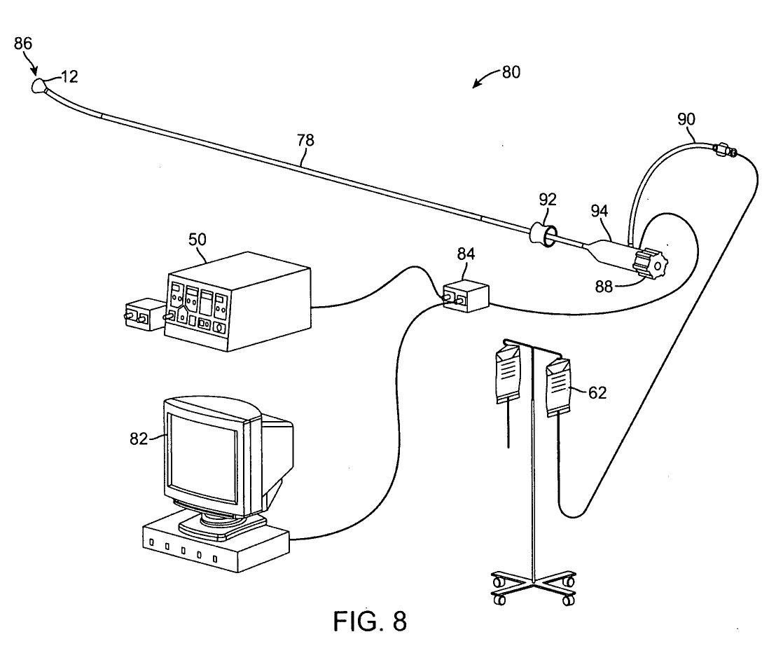

Another variation of a tissue imaging and treatment assembly 80 is illustrated in the perspective assembly view of FIG. 8, which shows an assembly which may be utilized in combination with an endoscope. Because assembly 80 may receive an endoscope through a lumen defined therethrough, the endoscope may provide imaging functionality as well as optional steering or articulation capabilities to the assembly 80 when in use in a patient. This allows for a system to be assembled which may be optionally disposed after a single use or limited number of uses. Accordingly, assembly 80 may generally comprise deployment catheter 78 which defines a lumen therethrough extending from hub 94. Hood 12 may be positioned upon the distal end of deployment catheter 78 and may optionally include an electrode assembly 86, e.g., mapping, pacing, and/or ablation electrodes, positioned upon the hood 12. Electrode assembly 86 may be electrically coupled through catheter 78 to a processor and/or video display 82, e.g., electrocardiogram (ECG) display, via junction 84, which may also be electrically coupled to generator 50 for providing power, e.g., RF energy, to electrode assembly 86. Hood 12 may be actuated between its low-profile delivery configuration and extended and deployed configuration via an actuating mechanism such as hood retraction control 92 which may be located along catheter 78. An optional fluid irrigation poll 90 may also extend from hub 94 to fluidly couple a reservoir 62, which may hold the clearing fluid (or other fluids), to hood 12 for providing the purging fluid.

Moreover, assembly 80 may also include interface seal 88 along hub 94 to provide a seal when an endoscope shaft is advanced through hub 94 and distally through catheter 78.

The electrode assembly 86 may comprise one or more electrodes positioned upon the distal membrane 40 of hood 12. These electrodes may be utilized, e.g., for pacing and/or mapping of electrophysiological signals of imaged tissue and/or lesion creation. Examples of electrodes or electrode systems which may be utilized with any of the catheter treatment systems described herein are described in further detail in U.S. patent application Ser. Nos. 11/848,532 filed Aug. 31, 2007; 12/118,439 filed May 9, 2008; 12/201,811 filed Aug. 29, 2008; 12/209,057 filed Sep. 11, 2008; and 12/268,381 filed Nov. 10, 2008, each of which is incorporated herein by reference in its entirety.

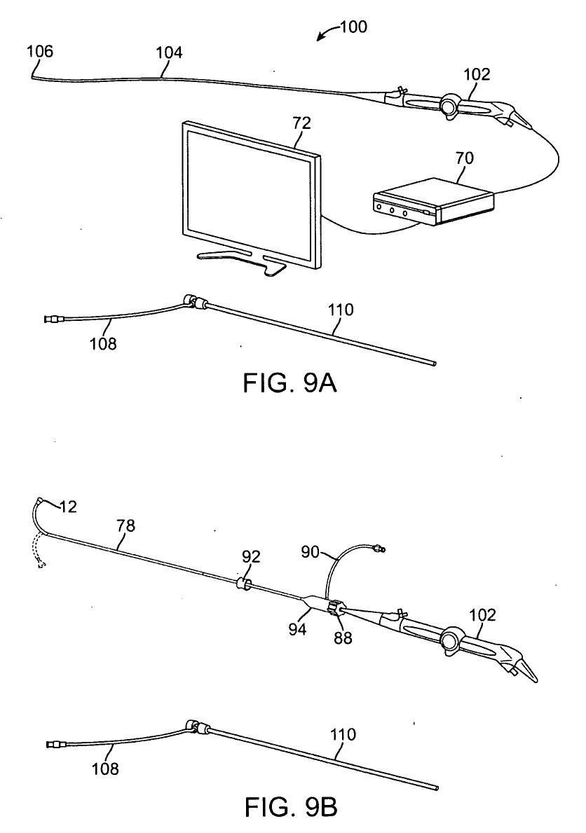

As previously mentioned, an endoscope may be inserted into the catheter system to optionally provide imaging functionality. An example of such an endoscopic assembly 100 is shown in the assembly view of FIG. 9A, which illustrates an endoscope having a handle 102 from which shaft 104 extends to an articulatable distal section having a distal end 106 with an integrated imaging assembly, e.g., fiberoptic, electronic CCD or CMOS imager, etc. Endoscope 100 may be coupled to video processor assembly 70 for projecting images captured from endoscope distal end 106 upon video display 72. Endoscope 100 may be a conventional device or it may alternatively be specially configured for use with the devices described herein.

In addition to endoscope 100, the deployment catheter assembly may be further utilized with introducer sheath 110 through which the catheter and endoscope 100 may be advanced. Introducer sheath 110 may further include a fluid irrigation port 108 extending from sheath 110 for coupling to a fluid reservoir or for providing access to other instruments into the patient body.

FIG. 9B shows a perspective assembly view of an endoscope 100 shaft advanced through seal 88 and hub 94 and into position within deployment catheter 78. Endoscope 100 may be advanced within catheter 78 until the endoscope distal end 106 is positioned proximally of, within, or distally of hood 12. Interface seal 88 may allow for the insertion and integration of any number of imaging endoscopes, such as ones utilizing CMOS/CCD imaging sensors to be modularly integrated into the tissue visualization and treatment system by introduction through the seal 88. The endoscope 100 may be inserted through seal 88 and into catheter shaft 78 until the distal end of the endoscope is in the imaging hood 12, as previously mentioned, and/or in fluid communication with the purged saline within hood 12 in order to visualize tissue regions underlying the hood 12. The endoscope 100 may be securely held in place relative to the catheter 78 by seal 88, which may comprise, e.g., a Touhy borst seal, hemostasis valve, one-way flow valve or other type of seal. Saline or other translucent/transparent fluids such as plasma or Fluoroinert™, may be introduced either through irrigation port 90 or through a fluid lumen defined through the endoscope 100.

Moreover, hood 12 may be articulated and positioned relative to catheter 78, as shown, by actuating the steerable distal end of endoscope 100 which in turn may position hood 12. The portion of catheter 78 which is proximal to hood 12 may comprise a passively steerable segment which is flexible such that it conforms to the endoscope steering yet remains torquable and pusliable. Accordingly, such a flexible segment along catheter 78 may be fabricated from a number of biocompatible polymers (e.g.,, Chronoflex™, silicone, Pebax, etc.) reinforced with single or multiple stainless stain or nitinol wires (e.g., 0.004 to 0.015 inches in diameter) which may be embedded longitudinally or braided within the wall of the flexible segment. Other reinforcement members may include, e.g., polytetrafluoroethylene (PTFE), Kevlar® (E. I. du Pont de Nemours, Wilmington, Del.), silk threads, etc. The flexible and passively steerable segment may also be fabricated from bioinert metallic tubes (such as medical grade 316LVM stainless steel, nitinol or titanium) laser cut for customized flexibility and torquability or bio inert metallic coils coated with a thin-layer boot made of biocompatible polymeric heat shrink or Pebax coatings.

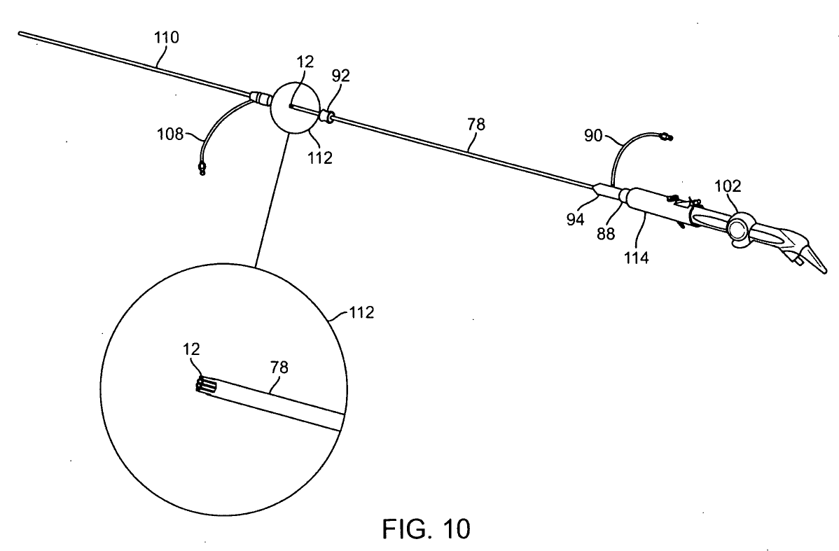

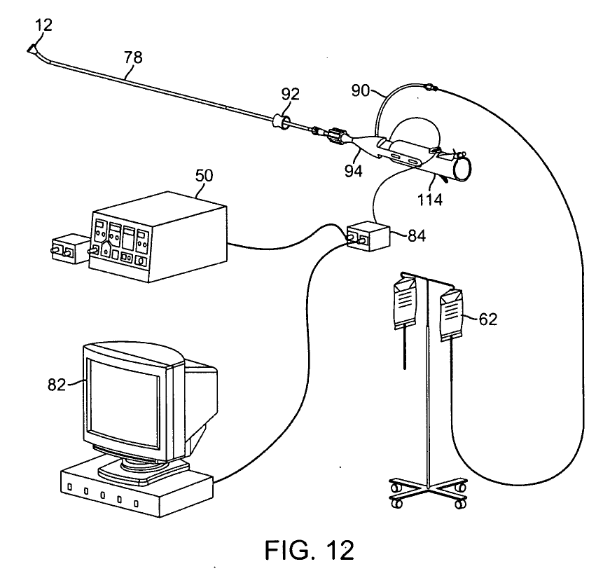

FIG. 10 illustrates a perspective assembly view of the endoscope 100 introduced within seal 88 and deployment catheter 78. Hood 12 may be seen in detail image 112 as having been retracted via hood retraction control 92 into its low-profile configuration within catheter 78. Hood 12 can be first collapsed hood retraction control 92 while saline is purged through hood 12 to ensure no bubbles are trapped inside hood 12. Catheter 78 may be advanced within introducer sheath 110 for deployment within the patient body. The variation shown illustrates an example where an additional endoscope handle interface 114 may be attached to hub 94 for facilitating coupling and de-coupling to endoscope handle 102. FIG. 12 also illustrates an assembly view which shows deployment catheter 78 coupled to hub 94, as above, but with the optional endoscope handle interface 114. Interface 114 may be configured to receive any number of endoscope handles for securely retaining and maintaining its position relative to catheter 78 when in use, described in further detail below.

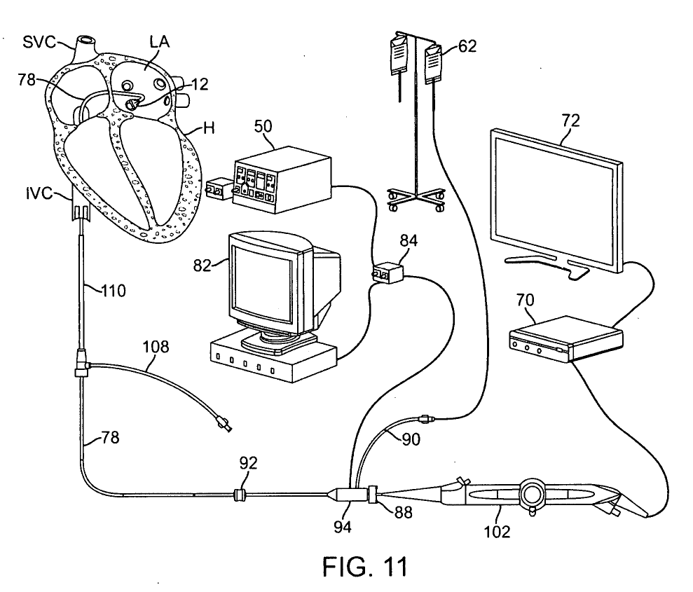

Turning now to FIG. 11, an illustrative assembly is shown of how a visualization catheter system may be configured and advanced intravascularly within a patient. Hood 12 and deployment catheter 78 may be advanced through introducer sheath 110 into the patient's vasculature, e.g., through the inferior vena cava IVC and transseptally into the left atrium LA of the patient's heart H, where tissue regions may be treated, such as lesion creation around the ostia of the pulmonary veins for treatment of atrial fibrillation. Once hood 12 has been advanced into the left atrium LA, hood 12 may be deployed to expand for visualization and tissue treatment. Hood 12 may be purged via saline fluid from reservoir 62 introduced through port 90 while the electrode assembly along hood 12 may be utilized to detect, e.g., ECG signals 82, or to ablate tissue via generator 50. The underlying tissue may be visualized via the endoscope imaging assembly which may capture and process the images for display upon monitor 72. Alternatively, hood 12 may be purged via fluid introduced through a fluid lumen defined through the endoscope itself.

The working channel of the endoscope and/or irrigation port can also be used to introduce guidewires, needles (such as transseptal or biologics delivery needles), dilators, ablation catheters (such as RF, cryo, ultrasound, laser and microwave), temperature monitoring probes, PFO closure devices, LAA closure implants, coronary artery stents, or other implantable devices or tools for performing diagnosis and/or treatment of the imaged target tissue. These lumens can also be used for the suction and/or evacuation of blood clots and/or any tissue debris as well as for the injection of contrast media for fluoroscopic imaging.

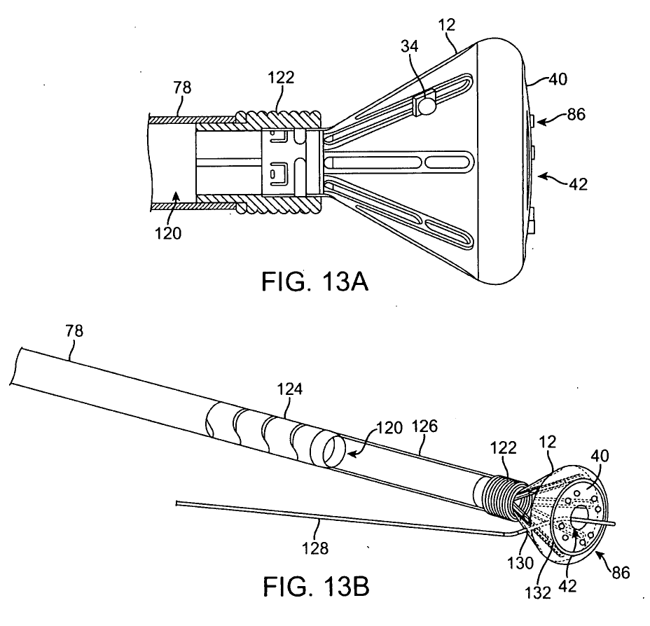

Turning now to the distal end of deployment catheter 78, FIG. 13A shows one example of a detailed cross-sectional side view of hood 12 coupled to catheter 78. Endoscope lumen 120 is illustrated as defined through catheter 78 which terminates in an opening into which conformable connector segment 122 may be disposed. Segment 122 may secure a proximal end of hood 12 thereto while defining a passage therethrough for allowing communication between an endoscope positioned within lumen 120 and an interior of hood 12. Segment 22 may be further configured with a conforming and bendable neck which may allow hood 12 to engage tissue perpendicularly when catheter 78 may be at an acute angle relative to a tissue surface. Such a conformable segment 22 may enable hood 12 to be placed in apposition against difficult-to-access tissue regions that may require torturous steering by catheter 78.

In this variation, hood 12 may comprise distal membrane 40, which defines aperture 42, and one or more electrodes 86 disposed over membrane 40. As previously mentioned, electrodes 86 may be utilized for pacing and/or mapping electrophysiological signals or for tissue ablation. Alternatively and/or additionally, the interiorly exposed struts along hood 12 may function as energy delivery electrodes to deliver RF energy through the conductive saline for virtual electrode ablation. Additionally, one or more light sources, such as light emitting diodes, may be mounted along the one or more support struts along hood 12 to provide off-axis illumination and glare prevention for illuminating the underlying tissue regions for imaging by imager 34.

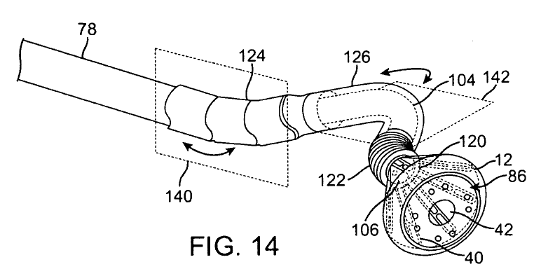

A perspective partial cross-sectional view is illustrated of hood 12 coupled via segment 122 to catheter 78 in FIG. 13B. The distal portion of catheter 78 may comprise an articulatable portion optionally having an articulatable segment 124 and/or a passively flexible segment 126 positioned distal to the articulatable segment 124. Articulatable segment 124 may be manipulated to move the distal segment, along with endoscope shaft 104 when positioned within lumen 120, within a first plane 140, as shown in FIG. 14. Steerable links may be provided along articulatable segment 124 to allow for articulation by manipulation of steering controls which may be found on the handle of the catheter 78. The steering links can also be steered by robotic control systems such as the Sensei™ Robotic Catheter System from companies such as Hansen Medical, Inc. (Sunnyvale, Calif.) or other robotic steering instruments.

The flexible segment 126 may further allow for the passive steering of hood 12 by conforming to the articulated endoscope 104 which may be moved within a second plane 142, which is different from the first plane 140, to provide additional degrees of freedom in steering and desirably positioning hood 12 relative to catheter 78 and the underlying tissue region. Further examples of actively and/or passively steered visualization catheters which may be utilized herein are described in further detail in the following U.S. patent application Ser. Nos. 12/108, 812 filed Apr. 24,2008; 12/117,655 filed May 8, 2008; and 12/209,057 filed Sep. 11, 2008, each of which is incorporated herein by reference in its entirety.

In addition to the steering capabilities of deployment catheter 78, hood 12 may utilize additional features such as a guidewire 128 which may pass through a rapid exchange port 130 defined along hood 12. Further examples of rapid exchange features which may be utilized with the systems herein are described in further detail in U.S. patent application Ser. No. 11/961,950 filed Dec. 20, 2007, which is incorporated herein by reference in its entirety. Yet another feature which may be optionally incorporated with hood 12 may include a ferromagnetic ring 132 for magnetic steering of the hood utilizing systems such as the Niobe® magnetic navigation system by Stereotaxis, Inc., which is further described in detail in U.S. patent application Ser. No. 11/848,532 filed Aug. 31, 2007, which is also incorporated herein by reference in its entirety.

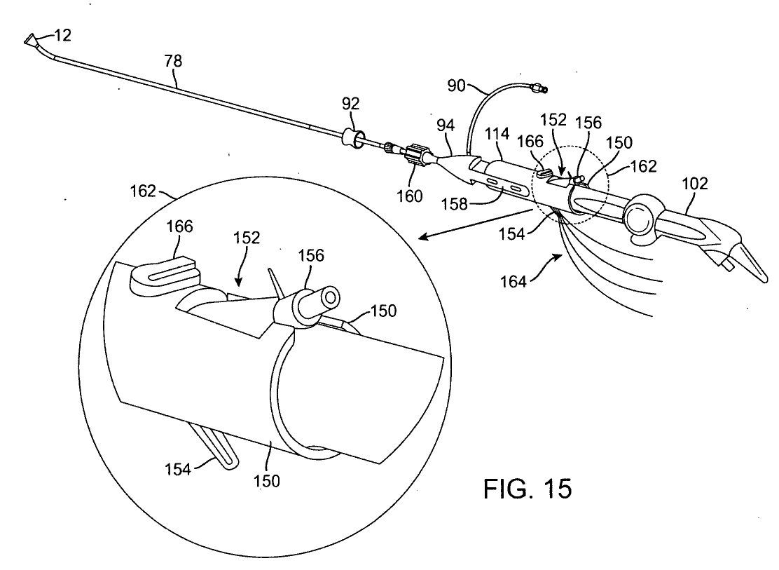

As previously described, an optional endoscope handle interface 114 may be attached to hub 94 for facilitating the coupling and de-coupling of catheter 78 to an endoscope handle 102, as shown in the perspective assembly view of FIG. 15. Interface 114 may be attached to hub 94 via one or more hub attachment members 158 and may further comprise a handle interface attachment 150 which allows for temporary securement of interface 114 to endoscope handle 102. Interface attachment 150 may generally comprise any number of mechanical fixtures for fitting interface 114 to endoscope handle 102, such as snap fit joints, screw joints between both handles, magnetic attachment using ferromagnetic components, clamps mounted on the interface 114 to clamp onto endoscope handle 102, Velcro® patches, etc., which may allow for interface 114 to be securely coupled to endoscope handle 102 and which may also allow for the de-coupling between the two for removal of the endoscope from catheter 78. One example shows attachment 150 configured as securement arm members which define an opening 152 to accommodate for the presence of an endoscope port 156 along endoscope handle 102, as shown in the detail view 162.

Interface 114 may further define at least one handle interface port 154 for coupling to, e.g., fluid lumen 164 or for allowing for the entry of other instruments such as a guidewire into catheter 78.

Additionally, articulation control 166, such as a knob, may be incorporated and positioned alone interface 114 for manipulating the articulatable segment 124 of catheter 78, as previously described. With deployment catheter 78 and the endoscope can be integrated, an operator may torque both the visualization catheter 78 and the endoscope by manipulating a single handle rather than two separate ones.

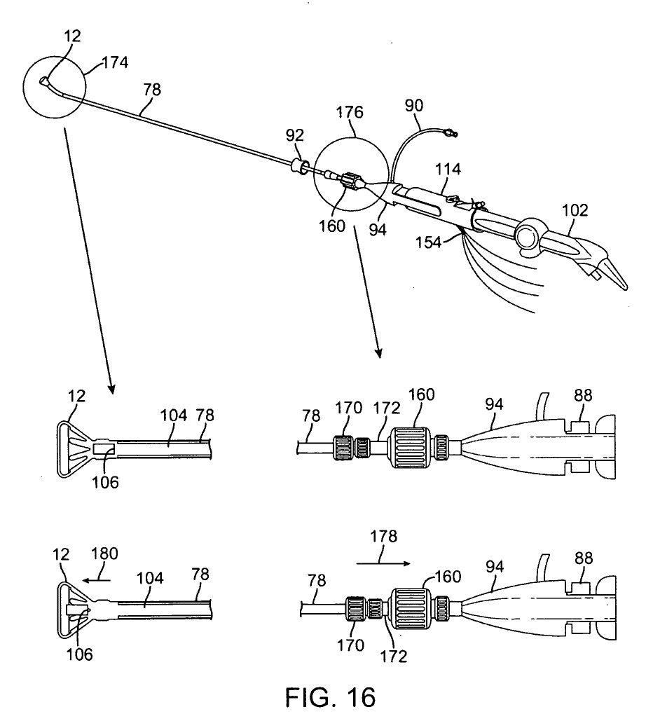

Another feature which may be optionally incorporated with deployment catheter includes advancement control 160, which may be positioned proximal to catheter 78. As illustrated in the perspective assembly and detail side views of FIG. 16, advancement control 160 may function as an optical zoom feature such that when control 160 is rotated about its longitudinal axis, the length of catheter shaft 78 may be varied relative to the length of the endoscope shaft 104 which in turn changes the relative position of the endoscope lens with respect to the imaging hood 12 and varies the distance between the lens and the imaged tissue.

As shown in the detail view of distal end 174 and detail view of proximal end 176 where endoscope shaft 104 is positioned within lumen 120 of catheter 78, if the distal end 106 of endoscope is initially positioned proximally of hood 12, rotation of control 160 in a first direction may shorten catheter shaft 78 by urging shaft control 170 to slide along coupler 172 towards control 160, as indicated by the proximal advancement 178 of shaft 78.

With endoscope shaft 104 maintained in its position by interface 114, the distal end 106 of endoscope may be positioned relatively closer to hood 12 and the underlying imaged tissue resulting in a zoom-in effect, as indicated by the relative distal advancement 180 of endoscope distal end 106. In the same manner, rotation of control 160 in a second direction opposite to the first direction may length catheter 78 to effectively move endoscope distal end 106 relatively farther from the underlying visualized tissue resulting in a zoom-out effect. Although the relative positioning of the endoscope distal end 106 relative to hood 12 and the underlying tissue may be effected by manually moving the endoscope relative to hood 12, use of control 160 allows for image adjustment in a controlled manner.

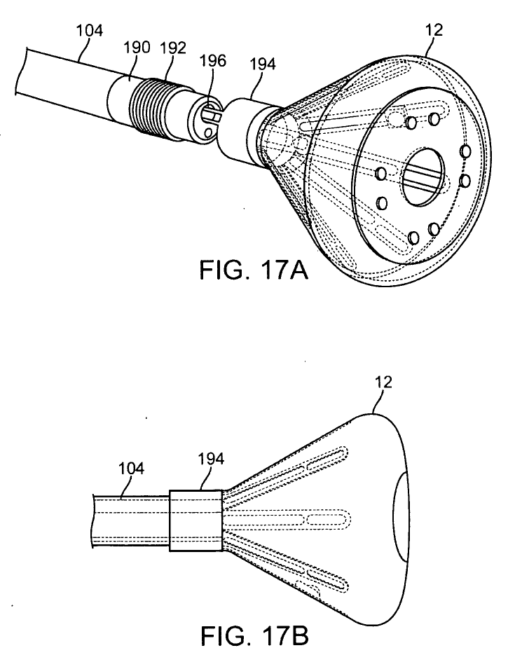

Turning now to other examples and features which may be utilized with the devices and methods described herein, hood 12 may be coupled directly to an endoscope distal end rather than utilizing a separate deployment catheter. As shown in the perspective and side views, respectively, of FIGS. 17A and 17B, a hood connector member 190 may be attached to a distal portion 190 of an endoscope shaft 104 via a securement portion 192 which defines a locking feature for coupling at least temporarily to hood 12, e.g., threaded as shown, tabs, screw-on coupler, male-female snap fits, elastic bands, clamps, friction lock, Velcro® patches, adhesive, etc. In this manner, securement portion 192 may be fitted upon any endoscope distal end by engaging with hood connector 194 located proximal to hood 12 in a complementary engagement. Any cables or connectors, such as wires attached to any electrodes or imaging sensors located within or along hood 12, leading from hood 12 may be passed through the endoscope working lumen 196 for coupling to their appropriate connections outside the patient body.

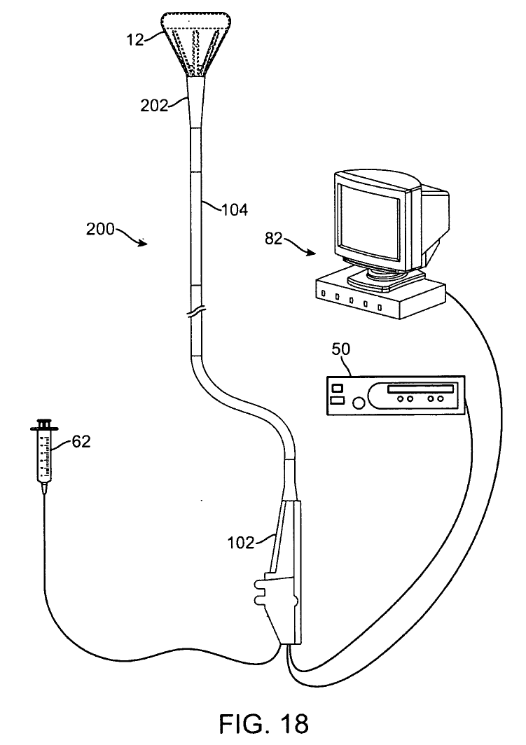

In yet another variation, the tissue visualization and ablation system may be configured as an end effector assembly which may be attachable or coupled to any number of other instruments. An example is shown in the assembly view of FIG. 18, which shows hood 12 having imaging element 34 self-contained as a separate assembly with a wire and/or connector bundle leading to an imaging element processor and/or display 82. The imaging hood assembly can be attached to the endoscope 200 by having attachment 202 affixed to the distal end of the endoscope 200, e.g., via usage of elastic bands, clamps, screws threads, slip-fit components, adhesive, sleeve couplers, etc. Saline or other transparent/translucent electrically conductive fluid, can be purged through the working channel of the endoscope 200. Other instruments (e.g., energy delivery probes, graspers, guidewires, ablation catheters, etc.) can also be advanced into the imaging hood via the working channel of the endoscope 200. Additionally, power generator 50 may provided for generating the ablation energy as well as an image processor and/or display 82 for viewing images either from an imaging element contained within or along hood 12 and/or as provided directly by the endoscope 200. Further examples of such devices are described in further detail in U.S. patent application Ser. No. 12/209,057 filed Sep. 11, 2008, which is incorporated herein by reference in its entilety.

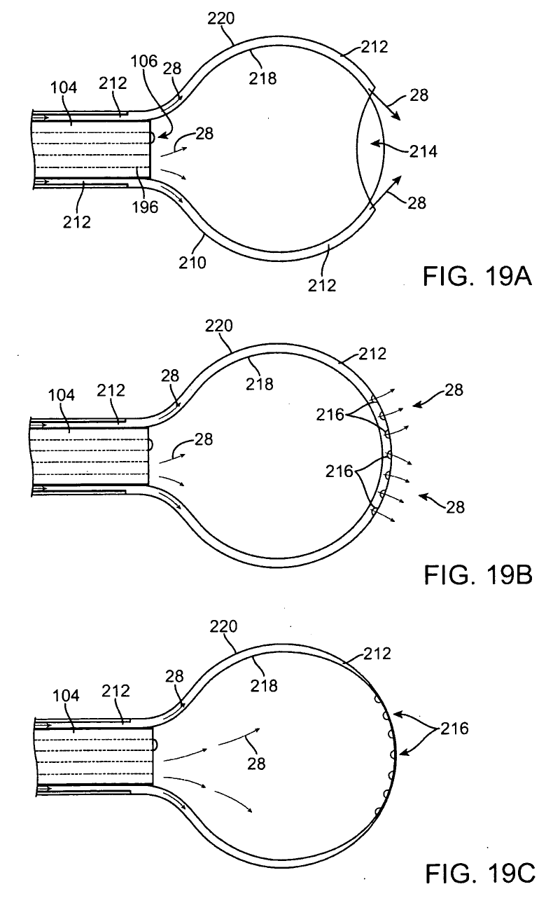

Aside from the use of hood structures, other imaging and treatment structures may be utilized with any of the deployment catheter devices described herein. FIG. 19A shows a partial cross-sectional side view of a double-layered balloon member 210 which defines an annular lumen 212 between an enclosed inner membrane 218 and an open outer membrane 220 through which the purging fluid 28 may be introduced as well as within the interior of balloon member 210. Balloon member 210 may be attached or coupled to an endoscope shaft 104, as previously described. As the inner and outer membranes 218, 220 may be fabricated from any number of transparent and distensible materials (e.g., polyethylene terephtlialate (PET), ChronoFlex™, ChronoPrene™, Nylon, latex, silicone, etc., or any of the other materials described above), the size of inner membrane 218 may be controlled by varying inflation pressure with the aid of a hydraulics pump, a peristaltic pump, or a pressurized intravenous bag, etc. The outer membrane 220 may also be controlled by a common pump or separately and may also define a single aperture or opening 214 through which saline may be purged to clear the viewing field when the inner membrane 218 is contacted against a tissue region to be imaged and/or treated.

In use, double-layered balloon member 210 may be advanced to establish physical contact on a tissue surface to be imaged. The purging fluid 28 may be pumped at a positive pressure through the annular lumen 212 until the fluid 28 fills said region completely and displaces any blood from within the aperture 214 and the interface between the outer membrane 220 and the tissue which the outer membrane 220 is in contact with. Fluid 28 may be pumped continuously or intermittently to allow for image capture by the imaging system of the endoscope. Fluid 28 purged from the outer membrane 218 may also be utilized for ablating the imaged tissue. Fluid 28, when in use, can conduct RF energy to the underlying tissue region. Moreover, cryogenic fluids, such as liquid nitrous oxide, may also be used in place of saline for cryo-ablation of tissue in contact.

In another variation, outer membrane 220 may define multiple apertures or openings 216 rather than a single aperture 214, as shown in the partial cross-sectional side views of FIGS. 19B and 19C. By manipulating the pressure of the fluid within the inner membrane 218 relative to tile pressure of the fluid within the annular lumen 212, the inner membrane 218 can be inflated to a pressure such that inner membrane 218 expands relative to outer membrane 220 and comes into contact against the outer membrane 220 to block the apertures 216 defined along outer membrane 220 and consequently preventing visualization/ablation fluid from flowing therethrough.

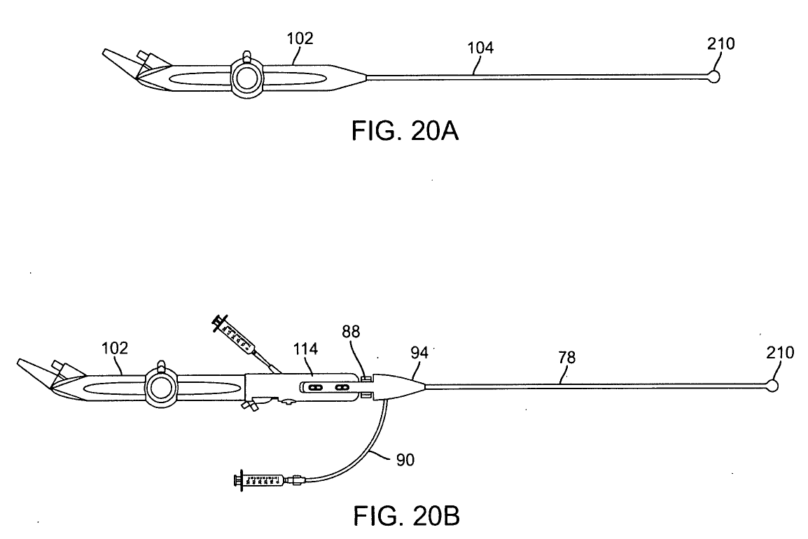

FIGS. 20A and 20B show side views of examples of an endoscope and tissue and treatment catheter combined with an endoscope, respectively, as previously described in combination with an inflatable double-layered balloon member 210 connected either directly. to endoscope shaft 104 or to deployment catheter 78.

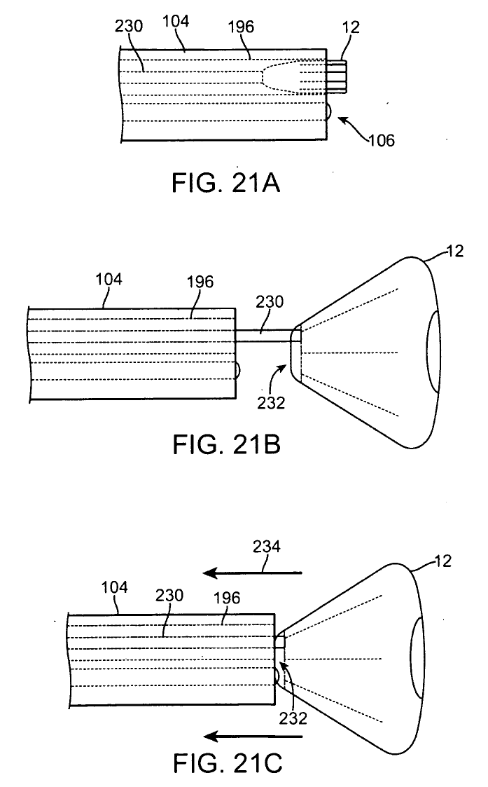

In yet another variation, FIGS. 21A to 21C illustrate a hood 12 which may be positioned upon a fluid support member 230 and advanced through an endoscope working lumen 196 while maintaining a low-profile delivery configuration. Upon advancement past the lumen opening, hood 12 may automatically expand or be actuated to expand into its deployed profile, as shown in FIG. 21B, such that a proximal hood opening 232 is defined through hood 12. Once expanded, support member 230 may be proximally withdrawn to pull hood 12 into firm contact against the distal end of endoscope shaft 104 such that opening 232 at least partially encircles the imaging element of the endoscope, as indicated by the direction of proximal withdrawal 234 in FIG. 21C. The interior of hood 12 may accordingly be purged of any blood by introducing the clearing fluid either through member 230 and/or endoscope lumen 196 for visualizing the underlying tissue, as described above. Moreover, hood 12 may be positioned off-axis relative to a central longitudinal axis of the endoscope shaft 104 to allow adequate space for the endoscope lens to engage and view through the proximal membrane of the hood.

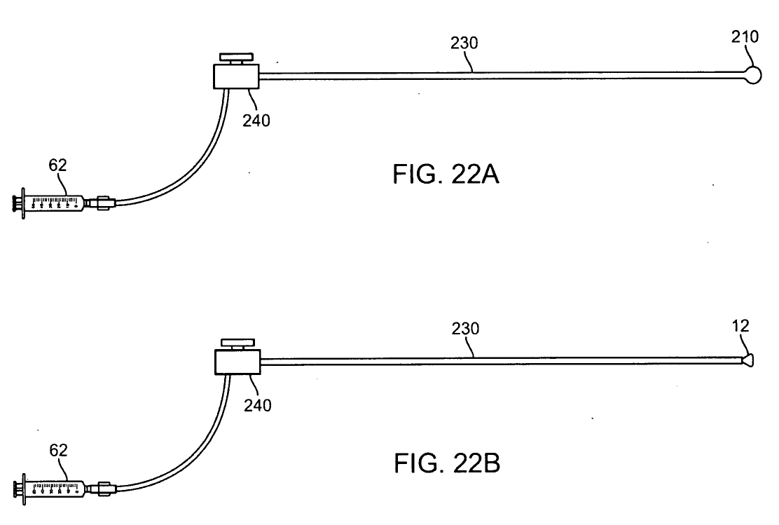

FIGS. 22A and 22B illustrate variations where either an inflatable balloon member 210 or hood 12 is shown, respectively, positioned upon support member 230 prior to insertion through an endoscope lumen. Tile proximal end of support member 230 may be coupled to hub 240, which may also be fluidly coupled to a fluid reservoir 62. Additionally, either balloon 210 or hood 12 may incorporate an imaging sensor directly within or along the assemblies for use alone or in combination with the imaging capabilities provided by the endoscope.

Any of the endoscopes used or accompanied with any of the systems described herein may include conventional endoscopes utilizing optical fiber imaging as well as endoscopes utilizing digital video platforms such as CMOS/CCD imagers for imaging under visible light. Additionally, other endoscopic imaging modalities such as infrared endoscopes, laser endoscopes, laparoscopes, etc. may alternatively be utilized as well.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A tissue treatment system, comprising:
a catheter defining a lumen therethrough and capable of intravascular delivery;
a hood attached to a distal end of the catheter such that the hood is reconfigirable between a low profile delivery configuration and a deployed configuration which defines an open area; and
an endoscope sized for insertion through the catheter lumen, wherein a distal end of the endoscope is positionable within or adjacent to the open area of the hood.

2. The system of claim 1 further comprising an imaging element within or along the hood such that the open area is contained within a visual field of the imaging element.

3. The system of claim 1 wherein the catheter further defines a fluid lumen therethrough in communication with the open area.

4. The system of claim 1 further comprising an electrode assembly disposed along the hood.

5. The system of claim 1 wherein the catheter comprises a steerable segment proximal to the hood.

6. The system of claim 1 wherein the catheter comprises a flexible segment which is conformable to a shape of the endoscope distal end.

7. The system of claim 1 wherein the catheter further comprises a hood retraction control operable to actuate the hood between the delivery configuration and the deployed configuration.

8. The system of claim 1 wherein the catheter further comprises an advancement control mechanism which is adapted to position the endoscope distal end between an advanced and retracted position relative to the hood.

9. The system of claim 1 further comprising a hub attached to a proximal end of the catheter.

10. The system of claim 9 further comprising a seal through which the endoscope is positioned.

11. The system of claim 1 further comprising an introducer sheath through which the catheter is advanceable.

12. The system of claim 1 further comprising a handle interface attached to a proximal end of the catheter, wherein the handle interface is coupled to a handle of the endoscope.

13. A method of deploying a tissue treatment system, comprising:
intravascularly advancing a catheter to a tissue region of interest;
reconfiguring a hood attached to a distal end of the catheter from a low-profile delivery configuration to a deployed configuration which defines an open area;
introducing a transparent fluid through the catheter and into the open area such that blood is cleared from the open area; and
adjusting a position of an endoscope distal end relative to the open area such that the tissue region of interest is visualized through the transparent fluid via the endoscope.

14. The method of claim 13 wherein intrasvascularly advancing further comprises advancing an introducer sheath positioned about the catheter.

15. The method of claim 13 wherein reconfiguring comprises actuating the hood between the delivery configuration and the deployed configuration via a hood retraction control positioned along the catheter.

16. The method of claim 13 wherein introducing comprises passing the transparent fluid through a lumen defined through the catheter and into the open area

17. The method of claim 13 wherein adjusting comprises retracting and/or advancing a length of the catheter relative to the endoscope.

18. The method of claim 13 further comprising articulating a segment of the catheter to reposition the hood relative to the tissue region of interest.

19. The method of claim 13 further comprising articulating a segment of the catheter via the endoscope positioned therewithin to reposition the hood relative to the tissue region of interest.

20. The method of claim 13 further comprising electrically sensing or detecting a physiological signal from the tissue region of interest via one or more electrodes positioned within or along the hood.

21. The method of claim 13 further comprising ablating the tissue region of interest via one or more electrodes positioned within or along the hood.
